(19) United States
(12) Patent Application Publication
(10) Pub. No.: US 2002/0198138 A1
(43) Pub. Date: Dec. 26, 2002

(54) Combination therapy for the treatment of inflammatory and respiratory diseases

(76) Inventor: William Louis Macias, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/149,366

(22) PCT Filed: Dec. 22, 2000
(86) PCT No.: PCT/US00/34261

(51) Int. Cl. A61K 38/17; A61K 31/22

(52) U.S. Cl. 514/2; 514/546

(57) ABSTRACT

A pharmaceutical composition for the treatment of Inflammatory Disease or Respiratory Disease in mammals, which comprises, as active ingredients, a neutrophil elastase inhibitor and Activated Protein C.

### FIELD OF THE INVENTION

This invention relates to the field of medicine and specifically to the treatment of Inflammatory Diseases and Respiratory Diseases.

### BACKGROUND OF THE INVENTION

Diseases of the respiratory system and inflammatory diseases present special problems for effective treatment. In particular, it is desirable to discover more effective treatments for diseases of the lower respiratory tract including the trachea, bronchi, and lungs. The greatest need is for new therapeutic agents to treat lung diseases and inflammatory diseases.

Present therapies for lower respiratory diseases and other inflammatory diseases are often only partially effective or are not suitable for extended use.

Lung diseases have been treated with neutrophil elastase inhibitors. For example, clinical trials have been conducted with the compound, Sivelestat, a neutrophil elastase inhibitor, (product of Ono Pharmaceutical Company, CAS No. 127373-66-4) for treatment of various lung disorders.

The prior art has also disclosed a relatively new pharmaceutical agent, “Activated Protein C,” 1 a serine protease, useful for treating sepsis (inclusive of severe sepsis).

It is desirable to create novel and more effective therapies for the treatment of respiratory diseases and inflammatory diseases.

### SUMMARY OF THE INVENTION

It is a discovery of this invention that respiratory and inflammatory diseases diseases are prevented or treated in an advantageous or superior manner by a combination therapy using (i) activated human Protein C and (ii) a neutrophil elastase inhibitor.

The combination therapy of activated human Protein C with an neutrophil elastise inhibitor synergistically improves treatment and prevention of respiratory or inflammatory diseases in the human body. Without being bound by any theory of operation, it is believed that the human body's response to the multi-faceted attack of both an anticoagulant and a neutrophil elastase inhibitor results in an increased efficacy of treatment or prevention, a decreased effective dosage, and/or a decreased duration of therapy.

This invention is a pharmaceutical composition comprising:

neutrophil elastase inhibitor, and Activated Protein C.

This invention is also a method of treating or preventing respiratory or inflammatory diseases by administering to a mammal in need thereof a therapeutically effective amount of (a) a neutrophil elastase inhibitor and a therapeutically effective amount of (b) Activated Protein C; wherein (a) and (b) are both administered within a therapeutically effective interval.

### DETAILED DESCRIPTION OF THE INVENTION

I. DEFINITIONS:

For purposes of the present invention, as disclosed and claimed herein, the following terms are as defined below:

aPC—Activated human Protein C, also called, Activated Protein C.

APTT—activated partial thromboplastin time.

hPC—human Protein C zymogen.

rhPC—recombinant human Protein C zymogen.

The terms “aPC,” “Activated human Protein C,” “Activated Protein C,” “raPC,” “recombinant Activated Protein C” are synonymous for the purpose and practice of this invention.

Protein C Activity—any property of activated human Protein C or its derivatives responsible for proteolytic, amidolytic, esterolytic, and biological (anticoagulant or pro-fibrinolytic) activities. Methods for testing for Protein C anticoagulant and amidolytic activity are well known in the art, i.e., see Grinnell et.al., 1987, Bio/Technology 5:1189-1192.

rhaPC—Recombinant activated human protein C, produced by activating r-HPC in vitro or by direct secretion of the activated form of Protein C from prokaryotic cells, eukaryotic cells, or from transgenic animals.

zymogen—an enzymatically inactive precursor of a proteolytic enzyme. Protein C zymogen, as used herein, refers to secreted, inactive forms, whether one chain or two chain, of protein C.

Respiratory Diseases—exemplified by lower respiratory diseases such as systemic inflammatory response syndrome, asthma, bronchitis, acute lung injury, acute resporatory distress syndrome, idiopathic pulmonary fibrosis, pneumonia, pulmonary edema, pulmonary obstructive disease, endotoxin induced lung damage, non-cell lung cancer, and multiple organ failure resulting from any of the preceding pathologic processes.

Inflammatory Diseases—refers to diseases such as inflammatory bowel disease, sepsis, septic shock, acuterespiratory distress syndrome, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, juvenile arthropathy or juvenile ankylosing spondylitis, reactive arthropathy, infectious or post-infectious arthritis, gonoccocal arthritis, tuberculous arthritis, viral arthritis, fungal arthritis, syphilitic arthritis, Lyme disease, arthritis associated with “vasculitic syndromes,” polyarteritis nodosa, hypersensitivity vasculitis, Luegenec's granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (typing), miscellaneous forms of arthritis, neuropathic joint disease (charco and joint), hemarthrosis (hemarthrosic), Henoch-Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, relapsing, and multiple organ failure resulting from any of the preceding pathologic processes.

The phrase “therapeutically effective amount” is an amount of (a) neutophil elastase inhibitor or an amount of (b) Activated Protein C which is effective to prevent or ameliorate respiratory disease.

The phrase “therapeutically effective interval” is a period of time beginning when one of either (a)the neutophil elastgase inhibitor or (b) Activated Protein C is administered to a mammal and ending at the limit of the beneficial effect in preventing or ameliorating respiratory or inflammatory disease or associated organ failure of (a) or (b).

The phrase “therapeutically effective combination,” used in the practice of this invention, means administration of both (a) neutrophil elastase inhibitor and (b) Activated protein C, either simultaneously or separately.

The term, “Active Ingredient” as used herein refers to a combination of (a) neutrophil elastase inhibitor and (b) Activated Protein C co-present in a pharmaceutical formulation for the delivery of a treatment regimen that applies this invention.

The term, “injectable liquid carrier” refers to a liquid medium containing either or both of (a) neutrophil elastase inhibitor, or (b) Activated Protein C; wherein (a) and (b) are independently dissolved, suspended, dispersed, or emulsified in the liquid medium.

II. Preparation of the Neutrophil Elastase Inhibitor Ingredient of the Invention.

The compositions and method of treatment of this invention use compounds known to be active as neutrophil elastase inhibitors. Preferred neutrophil elastase inhibitors are those disclosed in U.S. Pat. No. 5,017,610; 5,336,681; and 5,403,850, the disclosures of which are incorporated herein by reference. These patents also teach suitable method of making their respective inhibitors.

The neutrophil elastase inhibitors most preferred in the practice of this invention are those disclosed in U.S. Pat. No. 5,403,850. In particular, preferred inhibitors are those corresponding to formula (I)

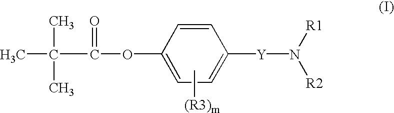

wherein Y represents sulfonyl (—SO₂—) or carbonyl;

(i) R1 and R2 which may be the same or different, each represent

(1) hydrogen,

(2) an alkyl of up to 16 carbon atoms or an alkyl of up to 16 carbon atoms substituted by carboxy,

(3) a group of the formula:

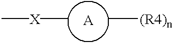

wherein

X represents a single-bond, sulfonyl (—SO₂—), an alkylene of up to 4 carbon atoms, or an alkylene of up to 4 carbon atoms substituted by —COOH or benzyloxy-carbonyl

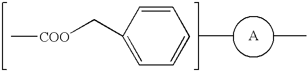

represents a carbocyclic ring or a heterocyclic ring, n represents an integer of 1 to 5,

R4 which may be the same or different represents,

(1) hydrogen or an alkyl group of up to 8 carbon atoms,

(2) an alkoxy of up to 14 carbon atoms,

(3) an alkylthio of up to 6 carbon atoms,

(4) hydroxy, halogen, nitro or trihalomethyl,

(5) a group of the formula: —NR41R42 wherein R41 and R42, which may be the same or different, each represents hydrogen or alkyl of up to 4 carbon atoms,

(6) tetrazole,

(7) sulfonic acid (—SO₃H) or hydroxymethyl (—CH₂OH),

(8) a group of the formula: —SO₂NR41R42 wherein R41 and R42 have the same meanings as described hereinbefore,

(9) a group of the formula: —Z41-COOR43 wherein Z41 represents a single-bond, an alkylene of up to 4 carbon atoms, or an alkenylene of from 2 to 4 carbon atoms, R43 represents hydrogen, an alkyl of up to 4 carbon atoms or benzyl,

(10) a group of the formula: —CONR41R42 wherein R41 and R42 have the same meanings as described hereinbefore,

(11) a group of the formula: —COO—Z42COOR43 wherein Z42 represents an alkylene of up to 4 carbon atoms, R43 represents hydrogen or an alkyl of up to 4 carbon atoms,

(12) a group of the formula: —COO—Z42-CONR41R42 wherein Z42, R41 and R42 have the same meanings as described hereinbefore,

(13) a group of the formula: —OCO—R45 wherein R45 represents an alkyl of up to 8 carbon atoms or p-guanidinophenyl,

(14) a group of the formula: —CO—R46 wherein R46 represents an alkyl of up to 4 carbon atoms,

(15) a group of the formula: —O—Z43-COOR45 wherein Z43 represents an alkylene of up to 6 carbon atoms, R45 represents a hydrogen atom, an alkyl group of up to 8 carbon atoms or a p-guanidinophenyl group,

(16) a group of the formula:

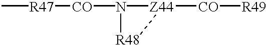

wherein —N—Z44-CO represents an amino acid residue, R48 represents hydrogen or alkyl of up to 4 carbon atoms, and R49 represents hydroxy, alkoxy of up to 4 carbon atoms, amino unsubstituted or substituted by one or two alkyls of up to 4 carbon atoms, carbamoylmethoxy unsubstituted or substituted by one or two alkyls of up to 4 carbon atoms at nitrogen of carbamoyl, R<47> represents a single-bond or an alkyl of up to 4 carbon atoms, or

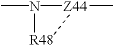

represents a heterocyclic ring containing 3 to 6 carbon atoms and R47 and R49 each has the same meaning as described hereinbefore,

(ii) R1, R2 and nitrogen bonded to R1 and R2 together represent a heterocyclic ring containing at least one nitrogen and substituted by —COOH, or an unsubstituted heterocyclic ring containing at least one nitrogen, R3 represents

(1) hydrogen,

(2) hydroxy,

(3) an alkyl of up to 6 carbon atoms,

(4) halogen,

(5) an alkoxy of up to 4 carbon atoms,

(6) an acyloxy of 2 to 5 carbon atoms, m represents an integer of up to 4, with the proviso that (1) when R1 and R2 represent hydrogen atom or alkyl group of up to 16 carbon atoms, and R3 represents a hydrogen atom or an alkyl group of up to 6 carbon atoms, Y represents carbonyl (—CO—), and that (2) the compounds wherein one of R1 and R2 represents hydrogen or an alkyl group of up to 16 carbon atoms or 2-carboxyethyl and the other of R1 and R2 represents a group of the formula:

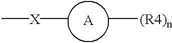

wherein X has the same meaning as described hereinbefore,

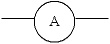

represents a pyridine or pyrrole ring, n represents an integer of 1 or 2, R4 which may be the same or different represents a hydrogen, an alkyl group of up to 8 carbon atoms or a group of the formula: —Z41-COOR43 wherein Z41 and R43 have the same meaning as described hereinbefore, m represents an integer of 1 or 2 and Y and R3 have the same meaning as described hereinbefore, are excluded, or pharmaceutically acceptable salts thereof.

Preferred compounds of formula (I) are those wherein wherein the amino acid-residue of R4 is a glycine-residue or an alanine-residue.

Specific highly preferred neutrophil elastase inhibitors having an R4 is a glycine-residue are as follows:

N-[o-(p-pivaloyloxybenzene)sulfonylaminobenzoyl]glycine,

N-[2-(p-pivaloyloxybenzene)sulfonylamino-5-chlorobenzoyl]glycine,

N-[5-methylthio-2-(p-pivaloyloxybenzene)sulfonylaminobenzoyl]glycine,

N-[2-(p-pivaloyloxybenzene)sulfonylamino-5-propylthiobenzoyl]glycine,

N-[5-methyl-2-(p-pivaloyloxybenzene)sulfonylaminobenzoyl]glycine, and

N-[o-(p-pivaloyloxybenzene)sulfonylaminobenzoyl]glycine methylester.

Specific highly preferred neutrophil elastase inhibitors having an R4 is a alanine-residue are as follows:

N-[o-(3-methyl-4-pivaloyloxybenzene)sulfonylaminobenzoyl]-d 1-alanine,

N-[o-(3-methyl-4-pivaloyloxybenzene)sulfonylaminobenzoyl]-beta-alanine,

N-[o-(e-methyl-4-pivaloyloxybenzene)sulfonylaminobenzoyl]-1-alanine,

N-[5-chloro-2-(3-methyl-4-pivaloyloxybenzene) sulfonylaminobenzoyl]-1-alanine and

N-[5-chloro-2-(3-methyl-4-pivaloyloxybenzene)sulfonylamino-benzoyl]-beta-alanine.

Most preferred is the compound represented by the structural formula (II):

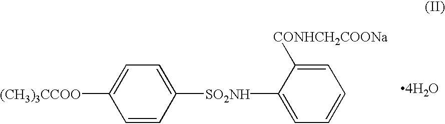

As acid addition salts of the compound of the general formula (I) are preferred non-toxic and water-soluble salts.

Suitable acid addition salts include, for example, an inorganic acid addition salt such as hydrochloride, hydrobromide, hydroiodide, sulfate, phosphate, nitrate, or an organic acid addition salt such as acetate, lactate, tartrate, benzoate, citrate, methanesulfonate, ethanesulfonate, benzenesulfonate, toluenesulfonate, isethionate, glucuronate, gluconate.

The compounds of the present invention of the general formula (I) may be converted into the corresponding salts by known methods. Non-toxic and water-soluble salts are preferable. Suitable salts, for example, are as follows:

salts of alkaline metal (sodium, potassium etc.), salts of alkaline earth metal (calcium, magnesium etc.), ammonium salts, salts of pharmaceutically acceptable organic amine (tetramethylammonium, triethylamine, methylamine, dimethylamine, cyclopentylamine, benzylamine, phenethylamine, piperidineamine, monoethanolamine, diethanolamine, tris (hydroxymethyl)amine, lysine, arginine, N-methyl-D-glucamine etc.).

Certain compounds of the invention may possess one or more chiral centers and may thus exist in optically active forms. Likewise, when the compounds contain an alkenyl or alkenylene group there exists the possibility of cis- and trans-isomeric forms of the compounds. The R- and S-isomers and mixtures thereof, including racemic mixtures as well as mixtures of cis- and trans-isomers, are contemplated by this invention. Additional asymmetric carbon atoms can be present in a substituent group such as an alkyl group. All such isomers as well as the mixtures thereof are intended to be included in the invention. If a particular stereoisomer is desired, it can be prepared by methods well known in the art by using stereospecific reactions with starting materials which contain the asymmetric centers and are already resolved or, alternatively by methods which lead to mixtures of the stereoisomers and subsequent resolution by known methods. For example, a racemic mixture may be reacted with a single enantiomer of some other compound. This changes the racemic form into a mixture of diastereomers and diastereomers, because they have different melting points, different boiling points, and different solubilities can be separated by conventional means, such as crystallization.

Prodrugs are derivatives of the compounds of the invention which have chemically or metabolically cleavable groups and become by solvolysis or under physiological conditions the compounds of the invention which are pharmaceutically active in vivo. Derivatives of the compounds of this invention have activity in both their acid and base derivative forms, but the acid derivative form often offers advantages of solubility, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Particularly preferred esters as prodrugs are methyl, ethyl, propyl, isopropyl, n-butyl, isobutyl, tert-butyl, morpholinoethyl, and N,N-diethylglycolamido.

N,N-diethylglycolamido ester prodrugs may be prepared by reaction of the sodium salt of a compound of Formula (I) (in a medium such as dimethylformamide) with 2-chloro-N,N-diethylacetamide (available from Aldrich Chemical Co., Milwaukee, Wis. USA; Item No. 25,099-6).

Morpholinylethyl ester prodrugs may be prepared by reaction of the sodium salt of a compound of Formula (I) (in a medium such as dimethylformamide) 4-(2-chloroethyl)morpholine hydrochloride (available from Aldrich Chemical Co., Milwaukee, Wis. USA, Item No. C4,220-3).

III. Preparation of the Activated Protein C Ingredient

Activated Protein C is a serine protease and naturally occurring anticoagulant that plays a role in the regulation of vascular homeostasis by inactivating Factors Va and VIIIa in the coagulation cascade. Human Protein C is made in vivo primarily in the liver as a single polypeptide of 461 amino acids.

In concert with other proteins, Protein C functions as an important down-regulator of blood coagulation factors that promote thrombosis. In other words, the Protein C enzyme system represents a major physiological mechanism of anticoagulation.

The critical role of protein C in controlling hemostasis is exemplified by the increased rate of thrombosis in heterozygous deficiency, protein C resistance (e.g., due to the common Factor V Leiden mutation) and the fatal outcome of untreated homozygous protein C deficiency. Human activated protein C, both plasma-derived and recombinant, have been shown to be effective and safe antithrombotic agents in a variety of animal models for both venous and arterial thrombosis. Activated protein C in recent clinical studies has been shown to be effective in human thrombotic diseases including the treatment of protein C deficiencies and microvascular thrombosis, such as disseminated intravascular coagulation associated with sepsis.

a. Preparation of Human Protein C

Recombinant human Protein C (r-hPC) was produced in Human Kidney 293 cells by techniques well known to the skilled artisan such as those set forth in Yan, U.S. Pat. No. 4,981,952, the entire disclosure of which is herein incorporated by reference. The gene encoding human Protein C is disclosed and claimed in Bang et al., U.S. Pat. No. 4,775,624, the entire disclosure of which is incorporated herein by reference. The plasmid used to express human Protein C in 293 cells was plasmid pLPC which is disclosed in Bang et al., U.S. Pat. No. 4,992,373, the entire disclosure of which is incorporated herein by reference. The construction of plasmid pLPC is also described in European Patent Publication No. 0 445 939, the teachings of which are also incorporated herein by reference and in Grinnell et al., 1987, Bio/Technology 5:1189-1192. Briefly, the plasmid was transfected into 293 cells, then stable transformants were identified, subcultured and grown in serum-free media. After fermentation, cell-free medium was obtained by microfiltration.

The human Protein C was separated from the culture fluid by an adaptation of the techniques of Yan, U.S. Pat. No. 4,981,952, the entire disclosure of which is herein incorporated by reference. The clarified medium was made 4 mM in EDTA before it was absorbed to an anion exchange resin (Fast-Flow Q, Pharmacia). After washing with 4 column volumes of 20 mM Tris, 200 mM NaCl, pH 7.4 and 2 column volumes of 20 mM Tris, 150 mM NaCl, pH 7.4, the bound recombinant human Protein C zymogen was eluted with 20 mM Tris, 150 mM NaCl, 10 mM CaCl2, pH 7.4. The eluted protein was greater than 95% pure after elution as judged by SDS-polyacrylamide gel electrophoresis.

Further purification of the protein was accomplished by making the protein 3 M in NaCl followed by adsorption to a hydrophobic interaction resin (Toyopearl Phenyl 650M, TosoHaas) equilibrated in 20 mM Tris, 3 M NaCl, 10 mM CaCl2, pH 7.4. After washing with 2 column volumes of equilibration buffer without CaCl2, the recombinant human Protein C was eluted with 20 mM Tris, pH 7.4. The eluted protein was prepared for activation by removal of residual calcium. The recombinant human Protein C was passed over a metal affinity column (Chelex-100, Bio-Rad) to remove calcium and again bound to an anion exchanger (Fast Flow Q, Pharmacia). Both of these columns were arranged in series and equilibrated in 20 mM Tris, 150 mM NaCl, 5 mM EDTA, pH 7.4. Following loading of the protein, the Chelex-100 column was washed with one column volume of the same buffer before disconnecting it from the series. The anion exchange column was washed with 3 column volumes of equilibration buffer before eluting the protein with 0.4 M NaCl, 20 mM Tris-acetate, pH 6.5. Protein concentrations of recombinant human protein C and recombinant activated Protein C solutions were measured by UV 280 nm extinction E0.1%=1.85 or 1.95, respectively.

b. Activation of Recombinant Human Protein C

Bovine thrombin was coupled to Activated CH-Sepharose 4B (Pharmacia) in the presence of 50 mM HEPES, pH 7.5 at 4° C. The coupling reaction was done on resin already packed into a column using approximately 5000 units thrombin/ml resin. The thrombin solution was circulated through the column for approximately 3 hours before adding MEA to a concentration of 0.6 ml/l of circulating solution. The MEA-containing solution was circulated for an additional 10-12 hours to assure complete blockage of the unreacted amines on the resin. Following blocking, the thrombin-coupled resin was washed with 10 column volumes of 1 M NaCl, 20 mM Tris, pH 6.5 to remove all non-specifically bound protein, and was used in activation reactions after equilibrating in activation buffer.

Purified rHPC was made 5 mM in EDTA (to chelate any residual calcium) and diluted to a concentration of 2 mg/ml with 20 mM Tris, pH 7.4 or 20 mM Tris-acetate, pH 6.5. This material was passed through a thrombin column equilibrated at 37(C with 50 mM NaCl and either 20 nM Tris pH 7.4 or 20 mM Tris-acetate pH 6.5. The flow rate was adjusted to allow for approximately 20 min. of contact time between the rHPC and thrombin resin. The effluent was collected and immediately assayed for amidolytic activity. If the material did not have a specific activity (amidolytic) comparable to an established standard of aPC, it was recycled over the thrombin column to activate the rHPC to completion. This was followed by 1:1 dilution of the material with 20 mM buffer as above, with a pH of either 7.4 or 6.5 to keep the aPC at lower concentrations while it awaited the next processing step.

Removal of leached thrombin from the aPC material was accomplished by binding the aPC to an anion exchange resin (Fast Flow Q, Pharmacia) equilibrated in activation buffer (either 20 mM Tris, pH 7.4 or 20 mM Tris-acetate, pH 6.5) with 150 mM NaCl. Thrombin does not interact with the anion exchange resin under these conditions, but passes through the column into the sample application effluent. Once the aPC is loaded onto the column, a 2-6 column volume wash with 20 mM equilibration buffer is done before eluting the bound aPC with a step elution using 0.4 M NaCl in either 5 mM Tris-acetate, pH 6.5 or 20 mM Tris, pH 7.4. Higher volume washes of the column facilitated more complete removal of the dodecapeptide. The material eluted from this column was stored either in a frozen solution (—20° C.) or as a lyophilized powder.

The anticoagulant activity of activated Protein C was determined by measuring the prolongation of the clotting time in the activated partial thromboplastin time (APTT) clotting assay. A standard curve was prepared in dilution buffer (1 mg/ml radioimmunoassay grade BSA, 20 mM Tris, pH 7.4, 150 mM NaCl, 0.02% NaN3) ranging in Protein C concentration from 125-1000 ng/ml, while samples were prepared at several dilutions in this concentration range. To each sample cuvette, 50 μl of cold horse plasma and 50 μl of reconstituted activated partial thromboplastin time reagent (APTT Reagent, Sigma) were added and incubated at 37° C. for 5 min. After incubation, 50 μl of the appropriate samples or standards were added to each cuvette. Dilution buffer was used in place of sample or standard to determine basal clotting time. The timer of the fibrometer (CoA Screener Hemostasis Analyzer, American Laboratory) was started immediately after the addition of 50 μl 37 (C 30 mM CaCl2 to each sample or standard. Activated Protein C concentration in samples are calculated from the linear regression equation of the standard curve. Clotting times reported here are the average of a minimum of three replicates, including standard curve samples.

IV. Pharmaceutical Compositions of the Invention

The pharmaceutical composition of the invention comprises as essential ingredients:

(i) neutrophil elastase inhibitor, and

(ii) Activated Protein C.

When these two ingredients are combined as a pharmaceutical composition the composition must be in a form which; (i) is itself in a liquid form suitable for administration by injection or, (ii) is in a form which is easily dissolved or suspended, or dispersed or emulsified into a liquid medium which is then suitable for administration by injection. When the pharmaceutical composition of the invention is prepared in injectable form it is a composition comprising as ingredients:

(a) a neutrophil elastase inhibitor,

(b) Activated Protein C, and

(c) an injectable liquid carrier.

a. Ratio and Amount of Ingredients in the Composition of the Invention

The essential ingredients (a) a neutrophil elastase inhibitor and (b) Activated Protein C are present in the formulation in such proportion that a dose of the formulation provides a pharmaceutically effective amount of each ingredient to the patient being treated.

The dose of composition of the invention to be administered is determined depending upon age, body weight, symptom, the desired therapeutic effect, the route of administration, and the duration of the treatment etc. Typically, the weight ratio of neutrophil elastase inhibitor to Activated protein C is from 1000:1 to 10000000:1 and preferably from 100:1 to 1000000:1.

An effective dosage of activated Protein C in human patients is considered to be between 0.1 and 2000 (micrograms/kg/day). Preferably, the dosage is between 1 and 1000 (micrograms/kg/day). A most preferred dosage of activated Protein C is between 100 and 1000 (micrograms/kg/day). A course of treatment is typically from 1 to 30 days.

For the neutrophil elastase inhibitor, in the human adult, the doses per person for intravenous therapy is from 1.0 to 5000 mg./day, and preferably from 250 to 500 mg./day. For oral administration the dose of neutrophil elastase inhibitor, is from 1.0 mg to 50,000 mg./day, and preferably from 500 to 5000 mg./day. A dose may be given continuously or intermittently (once or several times a day). A course of treatment is typically from 1 to 30 days.

In making compositions of the invention the essential ingredients; neutrophil elastase inhibitor and Activated Protein C are co-present and may be mixed in any homogeneous or non-homogeneous manner or adjacently or otherwise promixately placed together in an individual dosage unit suitable for practicing the method of the invention.

The dosage unit of the neutrophil elastase inhibitor will usually be admixed with a carrier or inert ingredients, or diluted by a carrier, or enclosed within a carrier which may be in the form of a ampoule, capsule, time release dosing device, sachet, paper or other container. When the carrier serves as a diluent, it may be a solid, semi-solid, paste, or liquid material which acts as a vehicle, or can be in the form of tablets, pills, powders, lozenges, elixirs, suspensions, emulsions, solutions, syrups, aerosols (as a solid or in a liquid medium), or ointment, containing, for example, up to 10% by weight of the active compound.

The dosage unit of the Activated Protein C will usually be admixed with a liquid carrier and/or other inert ingredients or enclosed within a carrier which may be in the form of a ampoule, bottle, time release dosing device or other container. When the carrier serves as a diluent, it may be a liquid material which acts as a vehicle, or can be in the form of solutions containing, for example, up to 10% by weight of the active compound. The Activated Protein C ingredient should be in an injectable liquid form immediately prior to use, however, it may be made in a storable form which is not a liquid but is easily convertable to a liquid (e.g., paste, liquid adsorbed on a solid, etc.)

For the pharmaceutical formulations containing both (a) neutrophil elastase inhibitor and (b) Activated Protein C the carrier may be an injectable liquid medium such as is well known in the art. The injectable liquid must be such that permits parenteral administration, that is, introduction of substances to a mammal being treated by intervenous, subcuataneous, intramuscular, or intramedullary injection. Intravenous injection is most preferred as a means of administration.

The Active ingredient can be dissolved or suspended in a pharmaceutically acceptable carrier, such as sterile water, sterile water containing saline and/or sugars and/or suspension agents or a mixture of both. For example, for intravenous injection the compounds of the invention may be dissolved in at a concentration of 2 mg./ml in a 4% dextrose70.5% Na citrate aqueous solution. Liquid compositions for oral administration include pharmaceutically-acceptable emulsions, solutions, suspensions, syrups and elixirs containing inert diluents commonly used in the art such as distilled water or ethanol. Besides inert diluents such compositions may also comprise adjuvants such as wetting and suspending agents, and sweetening, flavouring, perfuming and preserving agents.

Other compositions for oral administration include spray compositions which may be prepared by known methods and which comprise one or more of the active compound(s) Besides inert diluents such compositions may also comprise stabilizers such as sodium bisulfite and buffer for isotonicity, for example sodium chloride, sodium citrate or citric acid.

The manufacturing methods of spray compositions for inhalation therapy is described in detail, for example, in the specifications of U.S. Pat. No. 2,868,691 and U.S. Pat. No. 3,095,355.

Preparations for injection according to the present invention for parenteral administration include sterile aqueous or non-aqueous solutions, suspensions or emulsions. Example of aqueous solvents or suspending media are distilled water for injection and physiological salt solution. Examples of non-aqueous solvents or suspending media are propylene glycol, polyethylene glycol, vegetable oils such as olive oil, alcohols such as ehtanol, Polysorbate 80 (registered Trade Mark). These compositions may also include adjuvants such as preserving, wetting, emulsifying and dispersing agents stabilizing agents (e.g. lactose) and solubilizers (e.g. glutamic acid and asparaginic acid). They may be sterilized, for example, by filtration through a bacteria-retaining filter, by incorporation of sterilizing agents in the compositions or by irradiation. They may also be manufactured in the form of sterile solid compositions which can be dissolved in sterile water or some other sterile injectable medium immediately before use.

The neutrophil elastase inhibitor (when separate from the Activated Protein C) may be in the form of powder, tablet or capsule. A solid carrier can be one or more substances which may also act as flavoring agents, lubricants, solubilizers, suspending agents, binders, tablet disintegrating agents and encapsulating material. Suitable solid carriers are magnesium carbonate, magnesium stearate, talc, sugar lactose, pectin, dextrin, starch, gelatin, tragacanth, methyl cellulose, sodium carboxymethyl cellulose, low melting waxes, and cocoa butter.

The following pharmaceutical formulations are useful (as stated) for either the neutrophil elastase inhibitor alone, or the Active Ingredient which is a combination of (a) neutrophil elastase inhibitor and (b) Activated Protein.

Typically, from 10 mg to 1000 mg of the neutrophil elastase inhibitor is used in a unit dose of the formulation. The solution of the above Active Ingredient generally is administered intravenously to a subject at a rate of 1 ml per minute.

Typically, from 10 mg to 1000 mg of the Active Ingredient is used in a unit dose of the formulation.

A unit dosage formulation suitable for administration by continuous infusion is prepared by mixing at pH 6.0, Activated Protein C, a neutrophil elastase inhibitor, a salt (NaCl), a bulking agent (sucrose), and a buffer (citrate). The active ingredient, salt, and bulking agent are mixed in a weight to weight ratio of about 1 part Active ingredient, between about 7 and 8 parts salt, and between about 5 to 7 parts bulking agent. After mixing, the solution is transferred to vials and lyophilized. The vials comprising the active ingredients is sealed and stored until use.

V. Treating Respiratory Diseases and Inflammatory Diseases by The Method of the Invention

This invention is a method of treating or preventing Inflammatory Disease or Respiratory Disease by administering to a mammal in need thereof a therapeutically effective amount of (a) a neutrophil elastase inhibitor and a therapeutically effective amount of (b) Activated Protein C; wherein (a) and (b) are both administered within a therapeutically effective interval. The administration of (a) or (b) to a septic patient may be either continuous or intermittent.

A. Method of the Invention using Simultaneous Delivery of Activated Protein C and Neutrophil Elastase Inhibitor.

The Activated Protein C and a neutrophil elastase inhibitor can be delivered simultaneously. One convenient method of simultaneous delivery is to use the compositions of the invention described in section IV, supra, wherein the Active Ingredient has the essential ingredients co-present in a unit dosage form. Solutions or suspensions of mixed essential ingredients may, if desired, be delivered from the same IV liquid holding bag. Another method of simultaneous delivery of the Activated Protein C and a neutrophil elastase inhibitor is to deliver them to the patient separately but simultaneously. Thus, for example, the neutrophil elastase inhibitor may be given as an oral formulation at the same time the Activated Protein C is given parenterally. Dosage of a neutrophil elastase inhibitor can begin simultaneously with the activated Protein C administration. The length of the neutrophil elastase inhibitor administration can extend past the activated Protein C administration.

B. Method of the Invention using Non-Simultaneous Delivery of Activated Protein C and Neutrophil Elastase Inhibitor.

Each of the essential ingredients, viz., a therapeutically effective amount of (a) a neutrophil elastase inhibitor and a therapeutically effective amount of (b) Activated Protein C have a therapeutically effective interval, namely the interval of time in which each agent provides benefit for the patient being treated with Inflammatory Disease or Respiratory Disease. The method of the invention may be practiced by separately dosing the patient in any order with a therapeutically effective amount of (a) a neutrophil elastase inhibitor and a therapeutically effective amount of (b) Activated Protein C provided that each agent is given within the period of time that that the other agent is therapeutically effective against Inflammatory Disease or Respiratory Disease or organ failure resulting from these pathologic processes.

Typically, intravenous forms of neutrophil elastase inhibitor, for example, sodium N-[2-[4-(2,2-dimethylpropionyloxy)phenylsulfonyl-amino]benzoyl]aminoacetate tetrahydrate, are therapeutically effective immediately upon administration and up to 5 days later, and preferably in the time interval from 5 minutes after administration to 72 hours after administration. Similarly, salts of N-[2-[[[4-(2,2-dimethyl-1-oxopropoxy)phenyl]sulfonyl]amino]benzoyl]-glycine (CAS Registration No. 127373-66-4) may be used as oral forms of neutrophil elastase inhibitor and typically therapeutically effective from about 10 minutes to 5 days, and preferably from one-half hour to 72 hours after administration.

Dosage delivery of the neutrophil elastase inhibitor can begin up to 48 hours prior to the activated Protein C infusion with the preferred time being up to 24 hours and the most preferred being up to 12 hours. Alternatively, dosage of a neutrophil elastase inhibitor can begin up to 48 hours after the initiation of the activated Protein C infusion with the preferred time being up to 24 hours after and the most preferred being up to 12 hours after. The neutrophil elastase inhibitor can be administered by a variety of routes including oral, aerosol, rectal, transdermal, subcutaneous, intravenous, intramuscular, and intranasal, injectable solution. The activated Protein C compound can be administered as an injectable solution and by other routes including oral, aerosol, and intranasal. The Activated Protein C and neutrophil elastase inhibitor are preferably administered parenterally to a septic patient to insure their delivery into the bloodstream in an effective form as fast as possible.

VI. Duration of Treatment for Patients having Inflammatory Diseases or Respiratory Diseases using the Method of the Invention

The amount and relative ratio of Activated protein C and neutrophil elastase inhibitor to be used in the practice of the method of invention is set out in the previous section, (V) supra. It may be appreciated that it may be necessary to make routine variations to the dosage of either agent depending on the age and condition of the patient.

The decision to begin the therapy will be based upon the appearance of the clinical manifestations of Inflammatory Disease or Repiratory Disease. Typical clinical manifestations are coughing, restricted breathing, obstructed airway, pain, fever, chills, tachycardia, tachypnea, altered mental state, hypothermia, hyperthermia, accelerated or repressed breathing or heart rates, increased or decreased white blood cell count, and hypotension. For Respiratory Disease diagnostic tests such as roetgenographic examination, bronchoscopy, lung biopsy, spirography (lung capacity, residual volume, flow rates, etc.) are used. These and other symptoms and diagnostic techniques are well known in the art as set out in standard references such as, Harrison's Principles of Internal Medicine (ISBN 0-07-032370-4) 1994.

The decision to determine the length of therapy may be supported by standard clinical laboratory results from commercially available assays or instrumentation supporting the eradication of the symptoms defining Inflammatory or Respiratory Diseases. The method of the invention may be practiced by continuously or intermittently administering a therapeutically effective dose of the essential Activated Protein C and neutrophil elastase inhibitor ingredients for as long as deemed efficacious for the treatment of the septic episode. The administration can be conducted for up to a total of about 60 days with a preferred course of therapy lasting for up to 14 days.

The decision to terminate may also be based upon the measurement of the patient's baseline protein C levels returning to a value within the range of normal. The therapy may be restarted upon the return of the Inflammatory or Respiratory disease.

The combination therapy of activated Protein C and a neutrophil elastase inhibitor is also a safe and effective treatment in the prevention and treatment of pediatric forms of Disease.

While the present invention has been illustrated above by certain specific embodiments, it is not intended that these specific examples should limit the scope of the invention as described in the appended claims.

I claim:

1. A pharmaceutical composition comprising:
a neutrophil elastase inhibitor, and Activated Protein C.

2. A pharmaceutical composition of claim 1 wherein the neutrophil elastase inhibitor is represented by formula (I) 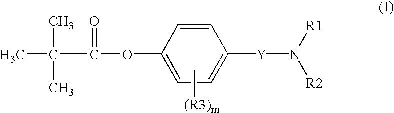
wherein Y represents sulfonyl (—SO₂—) or carbonyl;
(i) R1 and R2 which may be the same or different, each represent
(1) hydrogen,
(2) an alkyl of up to 16 carbon atoms or an alkyl of up to 16 carbon atoms substituted by carboxy,
(3) a group of the formula: 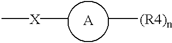
wherein
X represents a single-bond, sulfonyl (—SO₂—), an alkylene of up to 4 carbon atoms, or an alkylene of up to 4 carbon atoms substituted by —COOH or benzyloxy-carbonyl 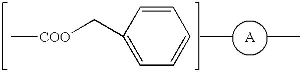
represents a carbocyclic ring or a heterocyclic ring, n represents an integer of 1 to 5,
R4 which may be the same or different represents,
(1) hydrogen or an alkyl group of up to 8 carbon atoms,
(2) an alkoxy of up to 14 carbon atoms,
(3) an alkylthio of up to 6 carbon atoms,
(4) hydroxy, halogen, nitro or trihalomethyl,
(5) a group of the formula: —NR41R42 wherein R41 and R42, which may be the same or different, each represents hydrogen or alkyl of up to 4 carbon atoms,
(6) tetrazole,
(7) sulfonic acid (—SO₃H) or hydroxymethyl (—CH₂OH),
(8) a group of the formula: —SO₂NR41R42 wherein R41 and R42 have the same meanings as described hereinbefore,
(9) a group of the formula: —Z41-COOR43 wherein Z41 represents a single-bond, an alkylene of up to 4 carbon atoms, or an alkenylene of from 2 to 4 carbon atoms, R43 represents hydrogen, an alkyl of up to 4 carbon atoms or benzyl,
(10) a group of the formula: —CONR41R42 wherein R41 and R42 have the same meanings as described hereinbefore,
(11) a group of the formula: —COO—Z42COOR43 wherein Z42 represents an alkylene of up to 4 carbon atoms, R43 represents hydrogen or an alkyl of up to 4 carbon atoms,
(12) a group of the formula: —COO—Z42-CONR41R42 wherein Z42, R41 and R42 have the same meanings as described hereinbefore,
(13) a group of the formula: —OCO—R45 wherein R45 represents an alkyl of up to 8 carbon atoms or p-guanidinophenyl,
(14) a group of the formula: —CO—R46 wherein R46 represents an alkyl of up to 4 carbon atoms,
(15) a group of the formula: —O—Z43-COOR45 wherein Z43 represents an alkylene of up to 6 carbon atoms, R45 represents a hydrogen atom, an alkyl group of up to 8 carbon atoms or a p-guanidinophenyl group,
(16) a group of the formula: 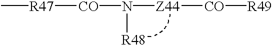
wherein —N—Z44-CO represents an amino acid residue, R48 represents hydrogen or alkyl of up to 4 carbon atoms, and R49 represents hydroxy, alkoxy of up to 4 carbon atoms, amino unsubstituted or substituted by one or two alkyls of up to 4 carbon atoms, carbamoylmethoxy unsubstituted or substituted by one or two alkyls of up to 4 carbon atoms at nitrogen of carbamoyl, R<47> represents a single-bond or an alkyl of up to 4 carbon atoms, or 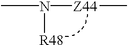
represents a heterocyclic ring containing 3 to 6 carbon atoms and R47 and R49 each has the same meaning as described hereinbefore,
(ii) R1, R2 and nitrogen bonded to R1 and R2 together represent a heterocyclic ring containing at least one nitrogen and substituted by —COOH, or an unsubstituted heterocyclic ring containing at least one nitrogen, R3 represents
(1) hydrogen,
(2) hydroxy,
(3) an alkyl of up to 6 carbon atoms,
(4) halogen,
(5) an alkoxy of up to 4 carbon atoms,
(6) an acyloxy of 2 to 5 carbon atoms, m represents an integer of up to 4, with the proviso that (1) when R1 and R2 represent hydrogen atom or alkyl group of up to 16 carbon atoms, and R3 represents a hydrogen atom or an alkyl group of up to 6 carbon atoms, Y represents carbonyl (—CO—), and that (2) the compounds wherein one of R1 and R2 represents hydrogen or an alkyl group of up to 16 carbon atoms or 2-carboxyethyl and the other of R1 and R2 represents a group of the formula: 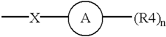
wherein X has the same meaning as described hereinbefore, 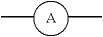
represents a pyridine or pyrrole ring, n represents an integer of 1 or 2, R4 which may be the same or different represents a hydrogen, an alkyl group of up to 8 carbon atoms or a group of the formula: —Z41-COOR43 wherein Z41 and R43 have the same meaning as described hereinbefore, m represents an integer of 1 or 2 and Y and R3 have the same meaning as described hereinbefore, are excluded, or pharmaceutically acceptable salts thereof.

3. A pharmaceutical composition of claim 1 wherein the neutrophil elastase inhibitor selected from the group consisting of:
N-[o-(p-pivaloyloxybenzene)sulfonylaminobenzoyl]glycine,
N-[2-(p-pivaloyloxybenzene)sulfonylamino-5-chlorobenzoyl]glycine,
N-[5-methylthio-2-(p-pivaloyloxybenzene)sulfonylaminobenzoyl]glycine,
N-[2-(p-pivaloyloxybenzene)sulfonylamino-5-propylthiobenzoyl]glycine,
N-[5-methyl-2-(p-pivaloyloxybenzene)sulfonylaminobenzoyl]glycine, and
N-[o-(p-pivaloyloxybenzene)sulfonylaminobenzoyl]glycine methylester,
N-[o-3-methyl-4-pivaloyloxybenzene)sulfonylaminobenzoyl]-d 1-alanine,
N-[o-(3-methyl-4-pivaloyloxybenzene)sulfonylaminobenzoyl]-beta-alanine,
N-[o-(e-methyl-4-pivaloyloxybenzene)sulfonylaminobenzoyl-1-alanine,
N-[5-chloro-2-(3-methyl-4-pivaloyloxybenzene)sulfonylaminobenzoyl]-1-alanine and
N-[5-chloro-2-(3-methyl-4-pivaloyloxybenzene)sulfonylamino-benzoyl]-beta-alanine.

4. A pharmaceutical composition of claim 1 wherein the neutrophil elastase inhibitor is N-{o-(p-pivaloyloxybenzene)sulfonylaminobenzoyl)glycine or salts, hydrated salts, or prodrug derivatives thereof.

5. The pharmaceutical composition of claims 1 wherein the weight ratio (a): (b) of (a) neutrophil elastase inhibitor and (b) Activated Protein C is 1000:1 to 10000000:1.

6. The pharmaceutical composition of claims 1 wherein the weight ratio (a): (b) of (a) neutrophil elastase inhibitor and (b) Activated Protein C is 100:1 to 1000000:1.

7. The pharmaceutical composition of claims 1 wherein the weight of (a) neutrophil elastase inhibitor is in the range of from 0.1 mg to 5000 mg and the weight of (b) Activated Protein C is in the range of 1.0 micrograms to 2000 micrograms.

8. The pharmaceutical composition of claims 1 comprising a suitable carrier, diluent or excipient therefor.

9. A method for the treatment or prevention of Inflammatory Disease comprising administering within a therapeutically effective interval to a mammal in need thereof, therapeutically effective amounts of; a neutrophil elastase inhibitor, and Activated Protein C.

10. A method for the treatment or prevention of Respiratory Disease comprising administering within a therapeutically effective interval to a mammal in need thereof, therapeutically effective amounts of;
a neutrophil elastase inhibitor, and Activated Protein C.

11. A method for treatment of a mammal to alleviate or prevent the pathological effects of Respiratory Disease, said method comprising administering to said mammal a therapeutically effective combination of Activated Protein C and a neutrophil elastase inhibitor represented by formula (I) 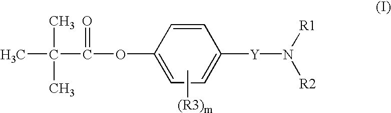
wherein Y represents sulfonyl (—SO₂—) or carbonyl;
(i) R1 and R2 which may be the same or different, each represent
(1) hydrogen,
(2) an alkyl of up to 16 carbon atoms or an alkyl of up to 16 carbon atoms substituted by carboxy,
(3) a group of the formula: 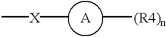
wherein
X represents a single-bond, sulfonyl (—SO₂—), an alkylene of up to 4 carbon atoms, or an alkylene of up to 4 carbon atoms substituted by —COOH or benzyloxy-carbonyl 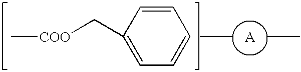
represents a carbocyclic ring or a heterocyclic ring, n represents an integer of 1 to 5,
R4 which may be the same or different represents,
(1) hydrogen or an alkyl group of up to 8 carbon atoms,
(2) an alkoxy of up to 14 carbon atoms,
(3) an alkylthio of up to 6 carbon atoms,
(4) hydroxy, halogen, nitro or trihalomethyl,
(5) a group of the formula: —NR41R42 wherein R41 and R42, which may be the same or different, each represents hydrogen or alkyl of up to 4 carbon atoms,
(6) tetrazole,
(7) sulfonic acid (—SO₃H) or hydroxymethyl (—CH₂OH),
(8) a group of the formula: —SO₂NR41R42 wherein R41 and R42 have the same meanings as described hereinbefore,
(9) a group of the formula: —Z41-COOR43 wherein Z41 represents a single-bond, an alkylene of up to 4 carbon atoms, or an alkenylene of from 2 to 4 carbon atoms, R43 represents hydrogen, an alkyl of up to 4 carbon atoms or benzyl,
(10) a group of the formula: —CONR41R42 wherein R41 and R42 have the same meanings as described hereinbefore,
(11) a group of the formula: —COO—Z42COOR43 wherein Z42 represents an alkylene of up to 4 carbon atoms, R43 represents hydrogen or an alkyl of up to 4 carbon atoms,
(12) a group of the formula: —COO—Z42-CONR41R42 wherein Z42, R41 and R42 have the same meanings as described hereinbefore,
(13) a group of the formula: —OCO—R45 wherein R45 represents an alkyl of up to 8 carbon atoms or p-guanidinophenyl,
(14) a group of the formula: —CO—R46 wherein R46 represents an alkyl of up to 4 carbon atoms,
(15) a group of the formula: —O—Z43-COOR45 wherein Z43 represents an alkylene of up to 6 carbon atoms, R45 represents a hydrogen atom, an alkyl group of up to 8 carbon atoms or a p-guanidinophenyl group,
(16) a group of the formula: 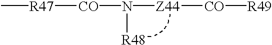
wherein —N—Z44-CO represents an amino acid residue, R48 represents hydrogen or alkyl of up to 4 carbon atoms, and R49 represents hydroxy, alkoxy of up to 4 carbon atoms, amino unsubstituted or substituted by one or two alkyls of up to 4 carbon atoms, carbamoylmethoxy unsubstituted or substituted by one or two alkyls of up to 4 carbon atoms at nitrogen of carbamoyl, R<47> represents a single-bond or an alkyl of up to 4 carbon atoms, or 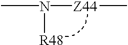
represents a heterocyclic ring containing 3 to 6 carbon atoms and R47 and R49 each has the same meaning as described hereinbefore,
(ii) R1, R2 and nitrogen bonded to R1 and R2 together represent a heterocyclic ring containing at least one nitrogen and substituted by —COOH, or an unsubstituted heterocyclic ring containing at least one nitrogen, R3 represents
(1) hydrogen,
(2) hydroxy,
(3) an alkyl of up to 6 carbon atoms,
(4) halogen,
(5) an alkoxy of up to 4 carbon atoms,
(6) an acyloxy of 2 to 5 carbon atoms, m represents an integer of up to 4,
with the proviso that (1) when R1 and R2 represent hydrogen atom or alkyl group of up to 16 carbon atoms, and R3 represents a hydrogen atom or an alkyl group of up to 6 carbon atoms, Y represents carbonyl (—CO—), and that (2) the compounds wherein one of R1 and R2 represents hydrogen or an alkyl group of up to 16 carbon atoms or 2-carboxyethyl and the other of R1 and R2 represents a group of the formula: 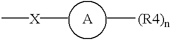
wherein X has the same meaning as described hereinbefore, 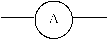
represents a pyridine or pyrrole ring, n represents an integer of 1 or 2, R4 which may be the same or different represents a hydrogen, an alkyl group of up to 8 carbon atoms or a group of the formula: —Z41-COOR43 wherein Z41 and R43 have the same meaning as described hereinbefore, m represents an integer of 1 or 2 and Y and R3 have the same meaning as described hereinbefore, are excluded, or pharmaceutically acceptable salts thereof.

12. The method according to claim 11 wherein the combination of Activated Protein C and a neutrophil elastase inhibitor is delivered parenterally.

13. The method according to claim 11, wherein the Activated Protein C is administered prior to the neutrophil elastase inhibitor.

14. The method according to claim 11 wherein the neutrophil elastase inhibitor is administered prior to the Activated Protein C.

15. Use of the composition of claim 1 for the manufacture of a medicament for treating Inflammatory Disease or Respiratory Disease in a mammal, including a human, currently afflicted with or susceptible to said Diseases.
